(19) United States
(12) Patent Application Publication
(10) Pub. No.: US 2022/0203368 A1
(43) Pub. Date: Jun. 30, 2022

(54) MICROFLUIDIC CHIP HAVING INCREASED THROUGHPUT FOR USE IN A SYSTEM FOR DELIVERY OF A PAYLOAD INTO A CELL

(71) Applicant: SQZ Biotechnologies Company, Watertown, MA (US)

(72) Inventor: Maisam DADGAR, Cambridge, MA (US)

(21) Appl. No.: 17/562,821

(22) Filed: Dec. 27, 2021

Related U.S. Application Data
(60) Provisional application No. 63/131,423, filed on Dec. 29, 2020.

(51) Int. Cl. B01L 3/00

(52) U.S. Cl. CPC B01L 3/502761; B01L 3/502707; B01L 2200/12; B01L 2300/0887; B01L 2200/0647; B01L 2300/0883; B01L 2400/086; B01L 2300/0816

(57) ABSTRACT

Provided is a microfluidic chip for causing the delivery of a payload to a cell comprising a first layer, a second layer, and a fluid flow region between the first layer and the second layer. This microfluidic chip is configured to accept flow of a cell suspension into the fluid flow region. The first layer of the microfluidic chip comprises a protrusion extending toward the second layer to form a constriction between the protrusion and the second layer, wherein the constriction is configured to cause perturbation of a cell membrane of a cell of the cell suspension as the cell passes through the constriction.

### CROSS-REFERENCE TO RELATED APPLICATION(S)

This application claims benefit claims the benefit of U.S. Provisional Application No. 63/131,423, filed Dec. 29, 2020, the entire contents of which are hereby incorporated herein by reference.

### FIELD

The present disclosure relates to systems for delivery of a payload into a cell, and more specifically to microfluidic chips having constrictions for causing perturbations of cell membranes to allow passage of a payload through the perturbed cell membrane.

### BACKGROUND

The controlled delivery of various materials into cells is important in the developing medical field of cell therapy. For example, various research and therapeutic applications may include the delivery of peptides, nucleic acids, proteins, small molecules, and nanomaterials through cell membranes and into cells. As discussed in WO2013059343, WO2015023982, PCT/US2015/058489, PCT/US2015/060689, and PCT/US2016/13113, constricting microfluidic channels may be used to deliver compounds and other payloads into cells. As disclosed in PCT/US18/66295, tabletop laboratory and/or clinical systems may be configured to force a cell suspension through a constriction cartridge, wherein the constriction cartridge houses one or more constriction-containing elements (e.g., a part, piece, device, component, or the like, such as a microfluidic chip or a filter) having constricting channels or constricting pores, in order to cause perturbations in the membranes of the cells in the cell suspension.

### SUMMARY OF THE INVENTION

As explained above, systems for intracellular payload delivery include systems configured to force cells of a cell suspension through one or more constrictions of a microfluidic chip in order to cause perturbations of the cell membranes. However, known systems for intracellular payload delivery are prone to clogging of cells in and around the constrictions, have insufficient throughput rates, and are not sufficiently straightforward and efficient to manufacture.

Accordingly, there is a need for improved systems, methods, and techniques for intracellular payload delivery, including a need for improved microfluidic chips having high throughput rates and low cell clogging rates. There is also a need for microfluidic chips having improved geometric configurations allowing for improved throughput and improved resistance to cell clogging or other failure, as well as improved ease and efficiency of manufacture. The systems, methods, and techniques disclosed herein may address one or more of these needs to improve the geometric configurations, throughput, resistance to clogging or other failure, and ease and efficiency of manufacture of microfluidic chips and/or systems, methods of use, and/or methods of manufacture.

Provided herein are high-throughput microfluidic chips for use in systems for intracellular payload delivery. The microfluidic chips disclosed herein have constrictions for perturbing the cell membrane of a cell in a cell suspension when the cell flows through the constriction. In particular, the microfluidic chips described herein allow for an increased throughput and decreased cell clogging than that of known microfluidic chips, such as those described above. Additionally, the microfluidic chips described herein are also manufactured using more consistent, straightforward, and efficient methods. Accordingly, the microfluidic chips for perturbing a cell membrane and introducing a payload into the cell may be easier to produce, may achieve a higher throughput, and may minimize the amount of cell clogging as compared to known microfluidic chips for intracellular delivery payload.

Specifically, the microfluidic chips disclosed herein include one or more constrictions positioned between an upstream fluid flow region and a downstream fluid flow region. This constriction is significantly longer than constriction regions of known microfluidic chips, allowing for more cells to pass through the constriction simultaneously. Accordingly, the constriction provided in microfluidic chips described herein provide a greater perturbed cell throughput. Microfluidic chips provided herein also minimize the occurrence of cell clogging at or near the constriction(s) due to the unique geometry of the disclosed chips, described in detail below.

In addition to a constriction for perturbing a cell membrane, the microfluidic chips provided herein also include a first layer and a second layer with a fluid flow region formed between the first layer and second layer through which a cell suspension and a payload is configured to flow. The first layer can comprise one or more protrusions extending from an inner surface of the first layer towards the second layer. A space between an end of the protrusion and the second layer defines the constriction described above. The height of the constriction is dependent on the diameter of a cell such that when the cell flows through the constriction, its membrane is perturbed. After perturbation, the payload can then enter the cell before the cell membrane closes. In some embodiments, a microfluidic chip may comprise a single protrusion that follows a serpentine path across a surface of the microfluidic chip. A serpentine path can, for example, provide a much longer constriction than those of known microfluidic chips, allowing for a greater cell throughput and minimal cell clogging, as explained above.

In some embodiments, provided is a microfluidic chip for causing the delivery of a payload to a cell, the chip comprising: a first layer; a second layer; and a fluid flow region between the first layer and the second layer, wherein the chip is configured to accept flow of a cell suspension into the fluid flow region, the cell suspension comprising a plurality of cells, the first layer comprises a protrusion extending toward the second layer to form a constriction between the protrusion and the second layer, wherein the constriction is configured to cause perturbation of a cell membrane of a cell of the plurality of cells as the cell passes through the constriction.

In some embodiments of the chip, the first layer comprises silicon.

In some embodiments of the chip, the second layer comprises glass.

In some embodiments of the chip, the protrusion extends away from an inner surface of the first layer in a height direction perpendicular to the inner surface of the first layer and comprises a proximal end that is adjacent to the inner surface of the first layer.

In some embodiments of the chip, the protrusion extends away from an inner surface of the first layer in a height direction perpendicular to the inner surface of the first layer and comprises a distal end of the protrusion that forms the constriction between the distal end of the protrusion and an inner surface of the second layer, wherein a height of the constriction between the distal end of the protrusion and the inner surface of the second layer, as measured in the height direction, is less than or equal to 5 microns.

In some embodiments of the chip, the height of the constriction is less than a diameter of the cell of the plurality of cells.

In some embodiments of the chip, the microfluidic chip comprises an inlet and an outlet, wherein the inlet is positioned at a first end of the chip and the outlet is positioned at a second end of the chip opposite the first end, wherein a distance between the inlet and the outlet extends in a direction perpendicular to an inner surface of the first layer.

In some embodiments of the chip, the proximal end of the protrusion has a thickness extending perpendicular to the height direction and extending from an upstream side of the protrusion to a downstream side of the protrusion, wherein the thickness of the proximal end of the protrusion is greater than or equal to 10 microns.

In some embodiments of the chip, the distal end of the protrusion has a thickness extending perpendicular to the height direction and extending from an upstream side of the constriction to a downstream side of the constriction, wherein the thickness of the distal end of the protrusion is greater than or equal to 5 microns.

In some embodiments of the chip, the protrusion has a length extending perpendicular to the height direction and extending along an interface between an upstream side of the constriction and a downstream side of the constriction, wherein the length is greater than or equal to 0.5 cm.

In some embodiments of the chip, the interface between the upstream side of the constriction and the downstream side of the constriction that comprises one or more of a curve and an angle.

In some embodiments of the chip, the interface between the upstream side of the constriction and the downstream side of the constriction forms a serpentine path.

In some embodiments of the chip, the serpentine path of the interface is perpendicular to the height direction at all locations along the serpentine path.

In some embodiments of the chip, the serpentine path comprises one or more right angles.

In some embodiments of the chip, a first dimension of the microfluidic chip extending perpendicular to a height direction that is perpendicular to a planar surface of one or both of the first and second layers is 18-24 mm.

In some embodiments of the chip, a second dimension of the microfluidic chip extending perpendicular to a height direction that is perpendicular to a planar surface of one or both of the first and second layers is 8-15 mm.

In some embodiments of the chip, a height dimension of the microfluidic chip extending in a height direction that is perpendicular to a planar surface of one or both of the first and second layers is 1000-1500 microns.

In some embodiments of the chip, the second layer contacts the first layer at a plurality of support pillars, wherein each support pillar of the plurality of support pillars extends from an inner surface of the first layer to the second layer.

In some embodiments of the chip, the first layer has a total height of 500-750 microns as measured in a height direction that is perpendicular to a planar surface of the first layer.

In some embodiments of the chip, the first layer has a minimum height of 450-650 microns, wherein the minimum thickness is measured in a height direction that is perpendicular to an inner surface of the first layer and wherein the minimum thickness is measured from an outer surface of the first layer to a nearest inner surface of the first layer that interfaces with the fluid flow region.

In some embodiments of the chip, the second layer has a thickness of 450-800 microns as measured in a height direction that is perpendicular to a planar surface of one or both of the first and second layers.

In some embodiments of the chip, an uppermost surface of the protrusion comprises a planar surface parallel to an inner surface of the first layer and perpendicular to a height direction in which the protrusion extends from the inner surface of the first layer.

In some embodiments of the chip, an uppermost surface of the protrusion comprises a planar surface parallel to an inner surface of the second layer and perpendicular to a height direction in which the protrusion extends from the inner surface of the first layer.

In some embodiments of the chip, a side surface of the protrusion extends upwards from an inner surface of the first layer at an angle.

In some embodiments of the chip, the side surface of the protrusion is angled at 50-60 degrees from the inner surface of the first layer.

In some embodiments of the chip, the side surface of the protrusion is angled at 54.7 degrees from the inner surface of the first layer.

In some embodiments of the chip, the cell suspension comprises a payload.

In some embodiments of the chip, the chip is configured to operate at a pressure of greater than or equal to 10 psi.

In some embodiments of the chip, a quotient of a cross-sectional area of the constriction to a perimeter of the constriction is greater than or equal to 0.5 microns.

In some embodiments, provided is a method of causing the delivery of a payload to a cell, the method comprising: receiving flow of a cell suspension into a fluid flow region of a microfluidic chip, the cell suspension comprising a plurality of cells; perturbing a cell membrane of a cell of the plurality of cells by causing the cell to flow through a constriction formed between a protrusion and a second layer of the microfluidic chip, the protrusion extending from an inner surface of a first layer of the microfluidic chip toward the second layer of the microfluidic chip, wherein the perturbation of the cell membrane allows entry of a payload into the cell.

In some embodiments of the method, perturbing a cell membrane of a cell of the plurality of cells by causing the cell to flow through a constriction formed between a protrusion and a second layer of the microfluidic chip comprises causing the cell to flow through the constriction at a flow rate of greater than or equal to 0.5 msec.

In some embodiments of the method, the microfluidic chip is configured to operate at a pressure of greater than or equal to 10 psi.

In some embodiments of the method, the cell suspension comprises the payload.

In some embodiments of the method, the method comprises causing the payload to come into contact with the cell suspension following perturbation of the cell membrane.

In some embodiments, provided is a method of fabricating a microfluidic chip for causing the delivery of a payload to a cell, the method comprising: etching into a first layer to form a recessed surface of the first layer and a protrusion of the first layer; and affixing a second layer to the first layer to form a microfluidic chip having a fluid flow region defined between the recessed surface and the second layer and a constriction defined between the protrusion and the second layer.

In some embodiments of the method, etching into a first layer to form a recessed surface of the first layer and a protrusion of the first layer comprises using wet or dry chemical etchants.

In some embodiments of the method, etching into a first layer to form a recessed surface of the first layer and a protrusion of the first layer comprises etching into the first layer to a depth of greater than or equal to 30 microns to define the fluid flow region.

In some embodiments of the method, etching into a first layer to form a recessed surface of the first layer and a protrusion of the first layer comprises etching into the first layer to a depth of less than or equal to 5 microns to form a distal end of the protrusion.

In some embodiments of the method, etching into a first layer to form a recessed surface of the first layer and a protrusion of the first layer comprises etching into the first layer to form a distal end of the protrusion, wherein the distal end of the protrusion has a thickness that extends perpendicular to a height direction that is perpendicular to a planar surface of the first layer, wherein the thickness of the distal end extends from an upstream side of the protrusion to a downstream side of the protrusion, wherein the thickness of the distal end of the protrusion is greater than or equal to 5 microns.

In some embodiments of the method, the protrusion has a length extending perpendicular to the height direction and extending along an interface between an upstream side of the constriction and a downstream side of the constriction, wherein the length is greater than or equal to 0.5 cm.

In some embodiments of the method, the interface between the upstream side of the constriction and the downstream side of the constriction that comprises one or more of a curve and an angle.

In some embodiments of the method, the interface between the upstream side of the constriction and the downstream side of the constriction forms a serpentine path.

In some embodiments of the method, the serpentine path of the interface is perpendicular to the height direction at all locations along the serpentine path.

In some embodiments of the method, the serpentine path comprises one or more right angles.

In some embodiments, any one or more of the features, characteristics, or elements discussed above with respect to any of the embodiments may be incorporated into any of the other embodiments mentioned above or described elsewhere herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a top view of a microfluidic chip, according to some embodiments;

FIGS. 2A illustrates an isometric view of cells flowing over a protrusion and through a constriction of a microfluidic chip, according to some embodiments;

FIG. 2B illustrates a side view of cells flowing over a protrusion and through a constriction of a microfluidic chip, according to some embodiments;

FIG. 2C illustrates a top view of cells flowing over a protrusion and through a constriction of a microfluidic chip, according to some embodiments;

FIG. 2D illustrates an exploded view of a cartridge holding chips for delivering a payload to a cell, according to some embodiments;

FIG. 3 illustrates a method of causing the delivery of a payload to a cell, according to some embodiments;

FIG. 4 provides flow path shear stress data for microfluidic chips described herein, according to some embodiments;

FIG. 5 provides flow path shear rate data for microfluidic chips described herein, according to some embodiments;

FIG. 6 shows a cross-sectional view of a cell flowing through a constriction formed by a protrusion of a microfluidic chip provided herein, according to some embodiments;

FIG. 7 illustrates a process of etching a layer of silicon to form a protrusion of a microfluidic chip, according to some embodiments;

FIG. 8 illustrates ghost constituency data for five different microfluidic chip geometries, according to some embodiments;

FIG. 9 illustrates total OVA 647 delivery percentage for five different microfluidic chip geometries, according to some embodiments;

FIG. 10 illustrates total OVA 647 mean fluorescence intensity for five different microfluidic chip geometries, according to some embodiments;

FIG. 11 illustrates total percentage of annexin V+ cells for five different microfluidic chip geometries, according to some embodiments;

FIG. 12 illustrates total of mean fluorescence intensity for annexin V+ cells for five different microfluidic chip geometries, according to some embodiments;

FIG. 13 illustrates OVA 647 delivery percentage by cell population for five different microfluidic chip geometries, according to some embodiments;

FIG. 14 illustrates OVA 647 mean fluorescence intensity by cell population for five different microfluidic chip geometries, according to some embodiments;

FIG. 15 illustrates percentage of annexin V+cells by cell population for five different microfluidic chip geometries, according to some embodiments; and

FIG. 16 illustrates mean fluorescence intensity for annexin V+cells by cell population for five different microfluidic chip geometries, according to some embodiments.

### DETAILED DESCRIPTION OF THE INVENTION

Described below are exemplary embodiments of microfluidic chips for use in systems for partially or fully automated intracellular payload delivery, as well as associated devices, systems, methods, and techniques.

Microfluidic chips provided herein can provide an increased throughput and decreased occurrence of cell clogging. Specifically, the total constriction region of the microfluidic chips provided herein is significantly greater than that of known microfluidic chips, which allows a greater number of cells to pass through and become perturbed by the constriction. Since more cells can be perturbed faster, the microfluidic chips provided herein can allow for increased output of cells having a delivered payload.

Additionally, constrictions of the microfluidic chips provided herein constrict the cells in a height direction measured from a bottom surface of a first layer of the microfluidic chip to a top surface of a second layer of the microfluidic chip. Thus, the constriction is formed between the first layer of the microfluidic chip (e.g., a top surface of a protrusion) and the second layer of the microfluidic chip. By contrast, many constrictions of microfluidic chips known in the art are fabricated by etching narrow channels into a layer of the microfluidic chip, such that a constriction is formed between side walls of the etched channel. The geometry of the constrictions of the microfluidic chips provided herein allow for a considerably larger constriction region than those known in the art, which in turn allows for more cells to be perturbed simultaneously and/or during a defined period of time than that of known microfluidic chips.

Provided below are definitions of a number of terms used herein, a description of microfluidic geometries for use in intracellular payload systems, methods of causing delivery of a payload to a cell using microfluidic chips described herein, flow characteristics of fluid flow through microfluidic chips provided herein, methods of fabricating microfluidic chips provided herein, and examples of cellular perturbation using microfluidic chips provided herein. Each topic is described in detail below.

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the singular form “a”, “an”, and “the” includes plural references unless indicated otherwise.

It is understood that aspects and embodiments of the invention described herein include “comprising,” “consisting,” and “consisting essentially of” aspects and embodiments.

It is further understood that the terms “includes,” “including,” “comprises,” and/or “comprising,” specify the presence of stated features, integers, steps, operations, elements, and/or components, but do not preclude the presence or addition of one or more other features, integers, steps, operations, elements, components, and/or groups thereof.

The term “if” may be construed to mean “when” or “upon” or “in response to determining” or “in response to detecting,” depending on the context.

The term “about” as used herein refers to the usual error range for the respective value readily known to the skilled person in this technical field. Reference to “about” a value or parameter herein includes (and describes) embodiments that are directed to that value or parameter per se.

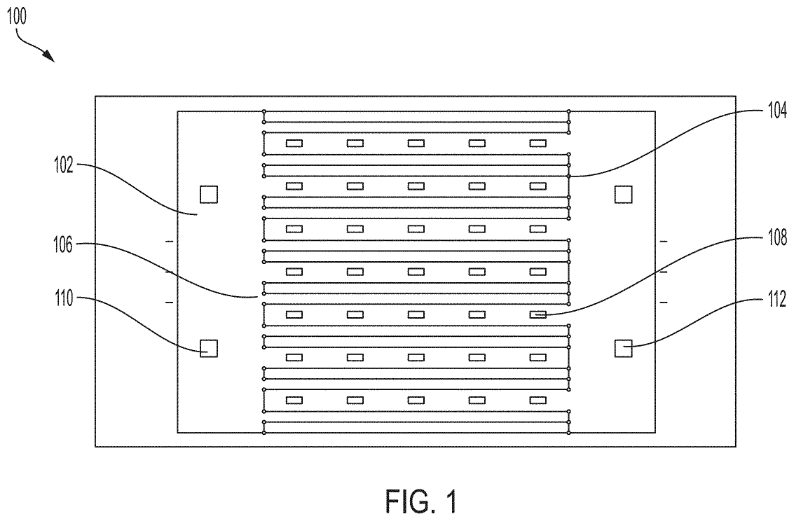

For purposes of the description herein, the directions and dimensions of a microfluidic chip may be referred to by the following convention: the x-direction or x-dimension may refer to the dimension running horizontally in FIG. 1 (the x-direction is the direction of overall flow of fluid through chip 100 of FIG. 1 from a flow inlet to a flow outlet); the y-direction or y-dimension may refer to the dimension running vertically in FIG. 1; and the z-direction or z-dimension may refer to the dimension running in and out of the page in FIG. 1.

Although the description herein uses terms first, second, etc. to describe various elements, these elements should not be limited by the terms. These terms are only used to distinguish one element from another.

For any of the structural and functional characteristics described herein, methods of determining these characteristics are known in the art.

All references cited herein, including patent applications and publications, are incorporated by reference in their entirety.

### Microfluidic Chip Geometries for Use in Intracellular Payload Delivery Systems

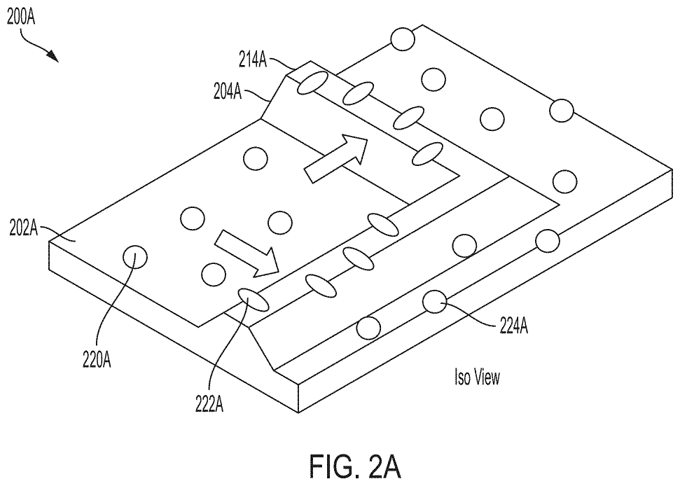

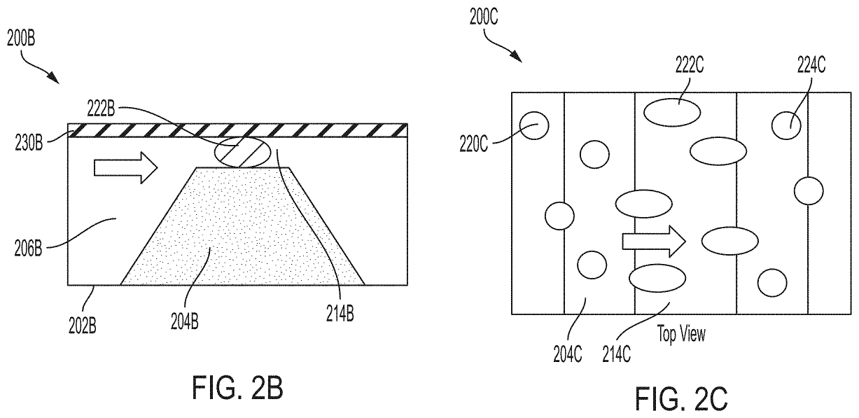

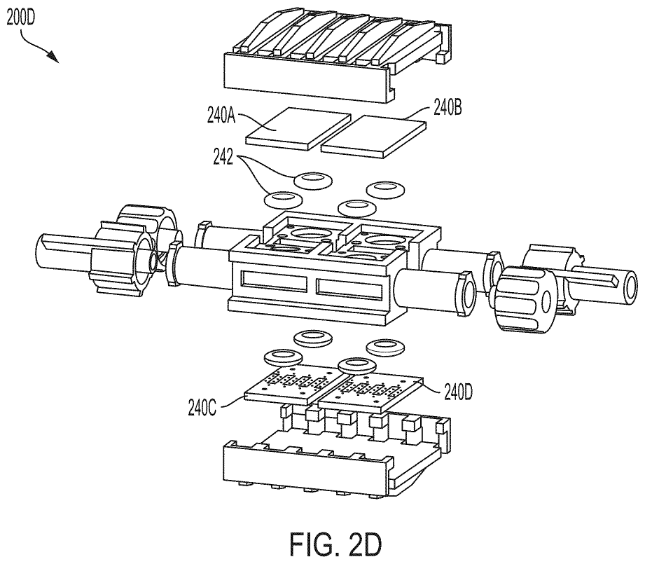

Described below are microfluidic chips for use in intracellular payload delivery systems that provide increased throughput and decreased cell clogging. Specifically, FIGS. 1-2C illustrate exemplary geometries of microfluidic chips provided herein. FIG. 1 shows a top view of a microfluidic chip, FIG. 2A shows an isometric view of a portion of a microfluidic chip (focusing on a protrusion and direction of fluid flow), FIG. 2B shows a side view of a portion of a microfluidic chip (focusing on a protrusion and constriction), and FIG. 2C shows a top view of a portion of a microfluidic chip (focusing on a constriction). Each is described in detail below.

FIG. 1 shows a top view of a microfluidic chip 100 according to some embodiments. Microfluidic chip 100 may be used in an intracellular payload delivery system. The features of intracellular payload delivery systems and devices are described more fully in PCT/US18/66295. In short, an intracellular payload delivery system may enable the delivery of a payload into cells by forcing the cells to flow through a constriction of a microfluidic chip, thereby perturbing the membranes of the cells and allowing the payload to enter the cells.

As shown in FIG. 1, microfluidic chip 100 may comprise first layer 102, protrusion 104, fluid flow region 106, support pillar 108, a fluid inlet 110, and a fluid outlet 112.

Microfluidic chip 100 is configured to receive a flow of a fluid at fluid inlet 110. Once the fluid enters microfluidic chip 100 at fluid inlet 110, the fluid flows through fluid flow region 106. Fluid flow region 106 may be defined by a first layer 102 of the microfluidic chip 100, a second layer of the microfluidic chip, and one or more protrusions 104.

In some embodiments, fluid inlet 110 is positioned at a first end of microfluidic chip 100 and fluid outlet 112 is positioned at a second end of microfluidic chip 100 opposite the first end. A distance between fluid inlet 110 and fluid outlet 112 can extend in a direction perpendicular to a planar surface (e.g., inner surface) of first layer 102. A distance between fluid inlet 110 and fluid outlet 112 may be 5-30 mm, 8-25 mm, or 10-20 mm. In some embodiments, the distance between fluid inlet 110 and fluid outlet 112 may be less than or equal to 450 mm, 250 mm, 100 mm, 50 mm, 30 mm, 25 mm, 20 mm, 15 mm, 10 mm, or 8 mm. In some embodiments, the distance between fluid inlet 110 and fluid outlet 112 may be greater than or equal to 5 mm, 8 mm, 10 mm, 15 mm, 20 mm, 25 mm, 30 mm, 50 mm, 100 mm, 250 mm, or 450 mm. In some embodiments, fluid inlet 110 and fluid outlet 112 of microfluidic chip 100 may be interchangeable. In some embodiments, fluid inlet 110 and/or fluid outlet 112 may be placed on the second layer. In some embodiments, fluid inlet 110 and/or fluid outlet 112 may be placed on first layer 102.

Microfluidic chip 100 operates under pressure to force the cells of the fluid to flow over the protrusion and through a constriction region defined by a distal end of the protrusion and the second layer of the microfluidic chip 100. The constriction/constriction region is described in more detail with respect to FIGS. 2A-2C.

First layer 102 comprises protrusion 104, which extends upward from an inner surface of first layer 102. Specifically, protrusion 104 extends from an inner surface of first layer 102 towards a second layer of microfluidic chip 100. Protrusion 104 comprises a proximal end, which is proximal to the inner surface of first layer 102, and a distal end, which is proximal to the second layer of microfluidic chip 100. A constriction is formed between the distal end of protrusion 104 and the second layer of microfluidic chip 100.

After the fluid flows through fluid flow region 106, passing over protrusion 104, the fluid exits microfluidic chip 100 by way of fluid outlet 112. When the fluid exits fluid flow outlet 112, it comprises a plurality of cells comprising payload.

Microfluidic chip 100 also comprises a plurality of support pillars 108. The second layer of microfluidic chip 100 are configured to contact first layer 102 only at the plurality of support pillars 108. In some embodiments, support pillars 108 can ensure that the second layer of microfluidic chip 100 is spaced apart from first layer 102 sufficiently to form the constriction(s). In some embodiments, an uppermost surface of support pillar 108 is higher than (or further from an outer surface of first layer 102 in a height direction extending perpendicular to a planar surface of first layer 102) an uppermost surface of protrusion 104.

A length, or a first dimension, of microfluidic chip 100 can extend perpendicular to a height direction of microfluidic chip 100 in the x-direction. The height direction extends perpendicular to a planar surface (e.g., an inner surface or an outer surface) of first layer 102. In some embodiments, the length of microfluidic chip 100 is 2-500 mm, 10-50 mm, 12-35 mm, 15-30 mm, or 18-25 mm. In some embodiments, the length of microfluidic chip 100 is greater than or equal to 2 mm, 4 mm, 10 mm, 20 mm, 30 mm, 40 mm, 50 mm, 100 mm, 250 mm, 450 mm, or 500 mm. In some embodiments, the length of microfluidic chip 100 is less than or equal to 2 mm, 4 mm, 10 mm, 20 mm, 30 mm, 40 mm, 50 mm, 100 mm, 250 mm, 450 mm, or 500 mm. A width, or a second dimension, of microfluidic chip 100 can also extend perpendicular to a height direction of microfluidic chip 100 in the y-direction. The height direction extends perpendicular to a planar surface (e.g., an inner surface or an outer surface) of first layer 102. In some embodiments, the width of microfluidic chip 100 is 2-100 mm, 5-50 mm, 5-25 mm, or 8-15 mm. In some embodiments, the width of microfluidic chip 100 is less than or equal to 2 mm, 4 mm, 10 mm, 20 mm, 30 mm, 40 mm, 50 mm, 100 mm, 250 mm, 450 mm, or 500 mm. In some embodiments, the width of microfluidic chip 100 is greater than or equal to 2 mm, 4 mm, 10 mm, 20 mm, 30 mm, 40 mm, 50 mm, 100 mm, 250 mm, 450 mm, or 500 mm.

A height dimension of microfluidic chip 100 extends in a height direction of microfluidic chip 100. The height direction extends perpendicular to a planar surface (e.g., an inner surface or an outer surface) of first layer 102. In some embodiments, the height of microfluidic chip 100 is less than or equal to 300 μm, 400 μm, 600 μm, 800 μm, 1 mm, 2 mm, 3 mm, or 5 mm. In some embodiments, the height of microfluidic chip 100 is greater than or equal to 300 μm, 400 μm, 600 μm, 800 μm, 1 mm, 2 mm, 3 mm, or 5 mm.

As explained above, microfluidic chip 100 is configured to receive flow of a fluid. The fluid may comprise a cell suspension. In some embodiments, the fluid may comprise a payload for delivery into a cell, wherein the payload may comprise any suitable cargo for delivery into a cell.

FIGS. 2A-2C illustrate various views of a portion of a microfluidic chip including a constriction and protrusion of a microfluidic chip (e.g., microfluidic chip 100 of FIG. 1) provided herein. FIG. 2A illustrates an isometric view of a portion microfluidic chip 200A including protrusion 204A and constriction 214A. A plurality of cells are shown flowing from an upstream region of the portion 200A of a microfluidic chip to a downstream region of microfluidic chip 200A. FIG. 2B illustrates a side view cross-section of a portion 200B of a microfluidic chip that shows a cell being perturbed as it flows through constriction 214B. FIG. 2C illustrates a top view of a portion 200C of a microfluidic chip that includes a plurality of cells flowing in a fluid flow region from an upstream region to a downstream region of portion 200C of a microfluidic chip, include some cells being perturbed as they pass through constriction 214C.

The portion 200A of a microfluidic chip shown in FIG. 2A further includes first layer 202A, an intact cell 220A, a squeezed cell 222A passing through constriction 214A, and cell 224A comprising a payload.

As shown in FIG. 2A, a plurality of cells is shown flowing in a fluid flow region from an upstream region of the fluid flow region to a downstream region of the fluid flow region. Specifically, intact cells 220A are located in the upstream region, and cells 224A comprising a payload are located in the downstream region. To pass from the upstream region to the downstream region, the cells must pass over protrusion 204A and through constriction 214A. Cells 222A are shown passing through constriction 214A. Constriction 214A is formed such that a height of constriction 214A (e.g., distance between the proximal end of protrusion 204A and the second layer of the microfluidic chip) is less than the diameter of a cell. Thus, as cell 222A passes through constriction 214A, it is squeezed such that its cell membrane is damaged or perturbed. This perturbation will allow intake of a payload in the downstream region of the microfluidic chip.

First layer 202A of the portion 200A of a microfluidic chip comprises protrusion 204A. As shown, protrusion 204A extends upward from an inner surface of first layer 202A. One or more sides of protrusion 204A may be angled. Protrusion 204A is defined by a proximal end proximate to first layer 202A and a distal end proximate to a second layer. In some embodiments, a width of the proximal end of protrusion 204A may be larger than a width of the distal end of protrusion 204A. In some embodiments, constriction 214A is defined by the distal end of protrusion 204A and a second layer of the microfluidic chip. In some embodiments, the distal end of protrusion 204A comprises a planar surface. The planar surface may be perpendicular to a height direction of the microfluidic chip, the height direction measured from an outer surface of first layer 204A to an outer surface of the second layer.

The planar surface of the distal end of protrusion 204A may have a width measured from the upstream side of protrusion 204A to the downstream side of protrusion 204A. This measurement may also be characterized as the width of the distal end of protrusion 204A. In some embodiments, the width may be 2-50 microns, 2-40 microns, 2-30 microns, 2-20 microns, 5-15 microns, or 8-12 microns. In some embodiments, the width may be less than or equal to 2 cm, 1 cm, 5 mm, 1 mm, 500 microns, 100 microns, 50 microns, 40 microns, 30 microns, 20 microns, 15 microns, 12 microns, 10 microns, 8 microns, or 5 microns. In some embodiments, the width may be greater than or equal to 2 microns, 5 microns, 8 microns, 10 microns, 12 microns, 15 microns, 20 microns, 30 microns, 40 microns, 100 microns, 500 microns, 1 mm, 5 mm, 1 cm, or 2 cm.

The proximal end of protrusion 204A may have a width greater than that of the distal end of protrusion 204A. The width of the proximal end of protrusion 204A may be measured from an upstream side of protrusion 204A to a downstream side of protrusion 204A. In some embodiments, the width may be 10-300 microns, 50-200 microns, 80-150 microns, or 100-125 microns. In some embodiments, the width of the proximal end of protrusion 204A may be less than or equal to 300 microns, 200 microns, 150 microns, 125 microns, 100 microns, 90 microns, 80 microns, or 50 microns. In some embodiments, the width of the proximal end of protrusion 204A may be greater than or equal to 10 microns, 50 microns, 80 microns, 90 microns, 100 microns, 125 microns, 150 microns, or 200 microns.

In some embodiments, the microfluidic chip comprises a single protrusion 204A having a plurality of segments or portions that extend in various directions across the inner surface of first layer 202A. In some embodiments, protrusion 204A extends in a direction that is perpendicular to a height direction of the microfluidic chip. (The height direction of the microfluidic chip runs perpendicular to a planar surface of an inner surface of the first layer.) In some embodiments, protrusion 204A extends along an interface between the upstream side and the downstream side of constriction 214A. In some embodiments, the length of single protrusion 204A (e.g., the total length of all segments/portions of protrusion 204A) is 0.5-200 cm, 5-175 cm, 25-150 cm, or 75-125 cm. In some embodiments, the length of single protrusion 204A is less than or equal to 200 cm, 175 cm, 150 cm, 125 cm, 100 cm, 75 cm, 50 cm, 25 cm, or 5 cm. In some embodiments, the length of single protrusion 204A is greater than or equal to 0.5 cm, 5 cm, 25 cm, 50 cm, 75 cm, 100 cm, 125 cm, 150 cm, or 175 cm.

As described above, the microfluidic chip may comprise a single protrusion 204A that extends in one or more directions across the inner surface of first layer 202A. For example, protrusion 204A may include a plurality of segments or portions such that two adjacent segments or portions extend in different directions across the inner surface of first layer 202A. In some embodiments, the microfluidic chip may comprise a plurality of protrusions 204A that extend in one or more directions across the inner surface of first layer 202A. In some embodiments, protrusion 204A extends along an interface between an upstream side of the constriction and a downstream side of the constriction. In some embodiments, the interface between the upstream side of the constriction and the downstream side of the constriction comprises a curve and/or an angle. In some embodiments, the interface between the upstream side of the constriction and the downstream side of the constriction forms a serpentine path. In some embodiments, the serpentine path of the interface is perpendicular to the height direction (e.g., running perpendicular to a planar surface of one or more of first layer 202A or a second layer) at all locations along the serpentine path. In some embodiments, the serpentine path of the interface between the upstream side of the constriction and the downstream side of the constriction comprises one or more right angles.

FIG. 2B illustrates a side view cross-section of a portion 200B of a microfluidic chip that shows a cell being perturbed as it flows through constriction 214B. The portion 200B of a microfluidic chip shown in FIG. 2B further includes first layer 202B, second layer 230B, fluid flow region 206B, protrusion 204B, constriction 214B, and squeezed cell 222B passing through constriction 214B. As squeezed cell 222B passes through constriction 214B, its cell membrane is damaged, or perturbed, to allow the intake of a payload through the perturbed cell membrane and into the cell.

Fluid flow region 206B is defined by an inner surface of first layer 202B, an inner surface of second layer 230B, and surfaces of protrusion 204B. The arrow depicted in FIG. 2B shows the direction of travel of cell 222B. Specifically, cell 222B travels from an upstream region of fluid flow region 206B, through constriction 214B, and to a downstream region of fluid flow region 206B.

In some embodiments, a distance between an inner surface of first layer 202B and an inner surface of second layer 230B as measured in a height direction of the microfluidic chip is 20-300 microns, 30-200 microns, or 50-100 microns. In some embodiments, the distance is less than or equal to 300 microns, 200 microns, 100 microns, 50 microns, or 30 microns. In some embodiments, the distance is greater than or equal to 20 microns, 30 microns, 50 microns, 100 microns, or 200 microns.

As shown, the cell travels through constriction 214B from an upstream side of protrusion 204B to a downstream side of protrusion 204B. This distance through constriction 214B is defined by a planar surface of the distal end of protrusion 204B. As described above with reference to FIG. 2A, this distance, or width of the distal end of protrusion 204B, may be 2-100 microns, 2-75 microns, 2-50 microns, 2-25 microns, 5-15 microns, or 8-12 microns. In some embodiments, the width of the distal end of protrusion 204B may be less than or equal to 100 microns, 75 microns, 50 microns, 25 microns, 15 microns, 12 microns, 10 microns, 8 microns, or 5 microns. In some embodiments, the width of the distal end of protrusion 204B may be greater than or equal to 2 microns, 5 microns, 8 microns, 10 microns, 12 microns, 15 microns, 25 microns, 50 microns, or 75 microns.

A total height of first layer 202B of the microfluidic chip, as measured in a height direction (the height direction running from the outer surface of first layer 202B to the outer surface of second layer 230B) may be characterized as measuring from the outer surface of first layer 202B to a topmost surface of a support pillar of first layer 202B (e.g., the only location at which second layer 230B contacts first layer 202B). In some embodiments, the total height of first layer 202B of the microfluidic chip may be 400-2000 microns, 450-800 microns, 500-750 microns, 550-700 microns, or 600-650 microns. In some embodiments, the total height of first layer 202B of the microfluidic chip may be less than or equal to 2000 microns, 1500 microns, 1000 microns, 800 microns, 750 microns, 700 microns, 650 microns, 600 microns, 550 microns, 500 microns, or 450 microns. In some embodiments, the total height of first layer 202B of the microfluidic chip may be greater than or equal to 400 microns, 450 microns, 500 microns, 550 microns, 600 microns, 650 microns, 700 microns, 750 microns, 800 microns, 1000 microns, or 1500 microns.

A minimum height of first layer 202B of the microfluidic chip, as measured in a height direction (the height direction running from the outer surface of first layer 202B to the outer surface of second layer 230B) may be characterized as measuring from the outer surface of first layer 202B to a nearest inner surface of first layer 202B that interfaces with fluid flow region 206B. In some embodiments, the minimum height may be 200-1000 microns, 400-700 microns, 450-650 microns, 500-600 microns, or 525-575 microns. In some embodiments, the minimum height may be less than or equal to 1000 microns, 700 microns, 650 microns, 600 microns, 600 microns, 575 microns, 525 microns, 500 microns, 450 microns, or 400 microns. In some embodiments, the minimum height may be greater than or equal to 200 microns, 400 microns, 450 microns, 500 microns, 525 microns, 575 microns, 600 microns, 650 microns, 700 microns, or 800 microns.

A height of second layer 230B may be measured from an inner surface of second layer 230B to an outer surface of 230B. In some embodiments, the height of second layer 230B may be 400-2000 microns, 450-800 microns, 500-750 microns, 550-700 microns, or 600-650 microns. In some embodiments, the height of second layer 230B of the microfluidic chip may be less than or equal to 2000 microns, 1500 microns, 1000 microns, 800 microns, 750 microns, 700 microns, 650 microns, 600 microns, 550 microns, 500 microns, or 450 microns. In some embodiments, the height of second layer 230B of the microfluidic chip may be greater than or equal to 400 microns, 450 microns, 500 microns, 550 microns, 600 microns, 650 microns, 700 microns, 750 microns, 800 microns, 1000 microns, or 1500 microns.

A width of the distal end of protrusion 204B may include the measurements as described above with respect to protrusion 204A of FIG. 2A. A width of a proximal end of protrusion 204B may include the measurements as described above with respect to protrusion 204A of FIG. 2A.

A height of constriction 214B, as measured from a planar surface of the distal end of protrusion 204B to the inner surface of second layer 230B, should be less than a diameter of a cell of the fluid flowing through the microfluidic chip. Specifically, this height should be less than a diameter of a non-squeezed cell (e.g., a cell within the upstream region of fluid flow region 206 or within a downstream region of fluid flow region 206B). In some embodiments, the height of constriction 214B is 1-8 microns, 1.25-5 microns, 1.5-3 microns, 1.75-2.75 microns, or 2-2.5 microns. In some embodiments, the height of constriction 214B is less than or equal to 8 microns, 7 microns, 6 microns, 5 microns, 4 microns, 3.5 microns, 3.25 microns, 3 microns, 2.75 microns, 2.5 microns, 2 microns, 1.75 microns, 1.5 microns, or 1.25 microns. In some embodiments, the height of constriction 214B is greater than or equal to 1 micron, 1.25 microns, 1.5 microns, 1.75 microns, 2 microns, 2.25 microns, 2.75 microns, 3 microns, 3.25 microns, 3.5 microns, 4 microns, 5 microns, 6 microns, or 7 microns.

Constriction 214B may be characterized by a quotient of a cross-sectional area to a perimeter of the constriction. In some embodiments, the quotient of a cross-sectional area to a perimeter of the constriction is 0.5-5 microns. In some embodiments, the quotient of a cross-sectional area to a perimeter of the constriction is less than or equal to 5, 4, 3, 2.5, 2, 1.5, or 1 micron. In some embodiments, the quotient of a cross-sectional area to a perimeter of the constriction is greater than or equal to 0.5, 1, 1.5, 2, 2.5, 3, or 4 microns.

In some embodiments, one or more sides of protrusion 204B may be angled. In some embodiments, a base angle at which protrusion 204B extends from the inner surface of first layer 202B, between a side of protrusion 204B and a base of protrusion 204B, may be 40-70 degrees, 45-65 degrees, or 50-60 degrees. In some embodiments, the base angle may be less than or equal to 70 degrees, 65 degrees, 60 degrees, 55 degrees, 50 degrees, or 45 degrees. In some embodiments, the base angle may be greater than or equal to 40 degrees, 45 degrees, 50 degrees, 55 degrees, 60 degrees, or 65 degrees. In some embodiments, the base angle is 54.7 degrees. In some embodiments, both base angles of protrusion 204B are 54.7 degrees. In some embodiments, the base angle is dependent upon the microfluidic chip fabrication method. For example, the base angle may depend on a potassium hydroxide (KOH) etching method for etching into a silicon first layer 202B.

In some embodiments, first layer 202B and second layer 230B each comprise silicon, glass, a polymer (e.g., polystyrene (PS) polycarbonate (PC), polyvinyl chloride (PVC), cyclic olefin copolymer (COC), polymethylmethacrylate (PMMA) or polydimethylsiloxane (PDMS)), or any other suitable material. In some embodiments, first layer 202B and second layer 230B comprise the same material composition. In some embodiments, the material composition of first layer 202B is different from that of second layer 230B. In some embodiments, one layer (e.g., first layer or second layer) is glass and the other layer (e.g., first layer or second layer) is silicon. In such embodiments, the constriction is defined by anodic bonding between the glass and silicon. This anodic bonding does not affect the total height of the microfluidic chip.

FIG. 2C shows a top view of a portion 200C of a microfluidic chip. Specifically, FIG. 2C shows a top view of protrusion 204C and a plurality of cells passing from an upstream region of a fluid flow region to a downstream region of the fluid flow region by way of constriction 214C (as illustrated by the arrow). The portion 200C of a microfluidic chip shown in FIG. 2C includes protrusion 204C, constriction 214C, intact cell 220C, squeezed cell 222C, and cell 224C comprising a payload.

As shown in FIG. 2C, protrusion 204C is characterized by a planar surface which defines constriction 214C, as described above with reference to protrusion 204A of FIG. 2A and protrusion 204B of FIG. 2B. Opposing the planer surface is an inner surface of a second layer of the microfluidic chip. When the microfluidic chip operates under pressure, cells are forced from the upstream region, squeezed through constriction 214C, and into the downstream region. Constriction 214C is configured such that a distance (e.g., height) between the planar surface of the distal end of protrusion 204C and the inner surface of the second layer is less than a diameter of a non-squeezed cell (e.g., cell 220C located in an upstream region of a fluid flow region or cell 224C comprising a payload and located in a downstream region of the fluid flow region). Constriction 214C may have a height as described above with reference to constriction 214B of FIG. 2B.

In the example shown in FIGS. 2A-2C, constrictions 214A-214C are formed between a distal end of protrusions 204A-C and an inner surface of a second layer. Specifically, in FIG. 2B, constriction 214B is formed by the space between a planar surface of the distal end of protrusion 204B and the inner surface of second layer 230B. In that example in FIG. 2B, the constriction has a constant height—as defined as the distance between the planar surface of the distal end of protrusion 204B and the inner surface of second layer 230B—across its entire length in the direction of flow. Thus, as viewed from the angle shown in FIG. 2B, constriction 214B has a rectangular shape. However, in some additional or alternative embodiments, a constriction, (such as constriction 214B) may have a trapezoidal, curved, stepped, or otherwise irregular cross-sectional shape, as viewed from the overhead angle depicted in FIG. 2B.

For example, in some embodiments, rather than having a uniform cross-sectional constriction height (in the vertical as illustrated in FIG. 2B), a constriction may have a tapered shape such that its cross-sectional height in the vertical direction increases or decreases (linearly or otherwise) along the length (in the horizontal direction as illustrated in FIG. 2B) of the constriction. In some embodiments, the cross-sectional height of a tapering constriction may increase or decrease (from any of the cross sectional constriction heights given herein) along the length of the entire constriction by about 1%, 2%, 3%, 5%, 10%, 25%, 50%, 100%, 200%, or 500%.

In some embodiments, rather than having a uniform cross-sectional constriction height (in the vertical direction as illustrated in FIG. 2B), a constriction may have a multi-stage constriction height, wherein the constriction has a stepped shape in which a plurality of stages of the constriction (in the horizontal direction as illustrated in FIG. 2B) have differing cross-sectional heights from one or more of the other stages. For example, in some embodiments, a constriction may have two stages in which a first stage is narrower (e.g., has a shorter height) than a second stage, or in which the second stage is narrower (e.g., has a shorter height) than the first stage. Cross-sectional heights of constriction stages may increase monotonically in the direction of flow in the constriction, may decrease monotonically in the direction of flow of the constriction, or may both increase and decrease in the direction of flow of the constriction (for example, in the case of a constriction having three or more constriction stages). Stages may be differentiated from one another by steps (in the vertical direction as illustrated in FIG. 2B) formed in one constriction wall or in both constriction walls (e.g., in the planar surface of the distal end of protrusion 204B, and/or in the inner surface of second layer 230B). Steps between stages in a constriction may be formed as a right-angle step and/or may be formed by a tapered transition region between the steps.

In some embodiments, any one or more constriction stages may have a cross-sectional constriction height (e.g., in the vertical direction as illustrated in FIG. 2B) equal to any of the cross-sectional constriction heights disclosed herein. In some embodiments, adjacent constriction stages may have cross-sectional constriction heights that vary from one another by about 1%, 2%, 3%, 5%, 10%, 25%, 50%, 100%, 200%, or 500%d.

In some embodiments, any one or more constriction stages may have a constriction length (e.g., in the direction of flow) equal to any of the constriction lengths disclosed herein. Alternatively, a set of constriction stages forming an entire constriction may have lengths that sum to equal any of the constriction lengths disclosed herein.

In some embodiments in which adjacent constriction stages are separated by a tapering constriction transition region, the length (e.g., in the direction of flow) of the tapering constriction transition region may be equal to about 0.1%, 0.5%, 1%, 2%, 3%, 5%, 10%, 25%, 50%, or 100% of any of the constriction lengths disclosed herein.

In some embodiments, constrictions of non-uniform (e.g., tapering) cross-sectional height and/or constrictions having multiple stages of different cross-sectional heights may allow for the chip to extend the amount of time for which a cell passing through the constriction is subject to pressure from the constriction walls, thereby extending the amount of time that pores in the cell wall are open and increasing payload delivery efficiency and effectiveness. In some embodiments, a constriction having an initial narrower portion followed by a subsequent wider portion may allow for pores to be formed quickly and effectively in the initial portion and for the pores to be caused to remain open as the cell passes through the wider portion. Using a wider constriction portion subsequent to a narrower constriction portion may achieve effective payload delivery while increasing cell viability as compared to implementations in which the entire constriction has the narrower constriction width.

### Chips Disposed in a Cartridge

FIG. 2D shows cartridge 200D holding four chips 240A, 240B, 240C, 240D therein. Cartridge 200D may hold chips inside it and direct the flow of fluid to and from chips held therein. As shown by the exploded view of FIG. 2D, o-rings 242 may press against a chip and surround an inlet or outlet port of the chip, holding the chip in place and forming a seal around the inlet/outlet port and facilitating flow of fluid into or out of the chip without leaking.

### Methods of Causing the Delivery of a Payload to a Cell Using Microfluidic Chips Provided Herein

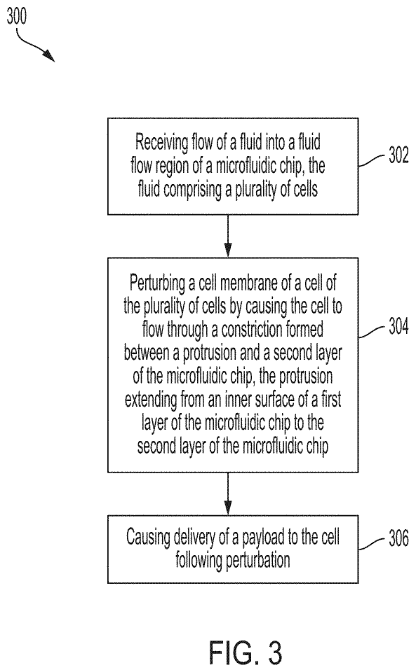

FIG. 3 shows an exemplary method 300 for causing the delivery of a payload to a cell using microfluidic chips described herein.

At step 302, the microfluidic chip (e.g., microfluidic chip 102 of FIG. 1) receives flow of a fluid into a fluid flow region. The fluid comprises a plurality of cells. In some embodiments, the fluid can also include a payload. Specifically, the microfluidic chip receives flow of a fluid in an upstream region of the fluid flow region.

At step 304, the microfluidic chip (e.g., microfluidic chip 102 of FIG. 1) causes perturbation of a cell membrane of a cell from the fluid by causing the cell to flow through a constriction in the microfluidic chip. The constriction is formed between a protrusion and a second layer of the microfluidic chip. The protrusion extends from an inner surface of a first layer of the microfluidic chip to the second layer of the microfluidic chip.

At step 306, delivery of a payload to the perturbed cell is caused. This occurs in a downstream region of the fluid flow region. In some embodiments, the payload is introduced following perturbation, at the downstream region of the fluid flow region. After delivery of the payload, the cell membrane may heal.

In some embodiments, cells processed through the constriction of a microfluidic chip described herein, for example in accordance with method 300, may demonstrate a viability percentage following passage through the constriction of 70-100%, 80-100%, 90-100%, or 95-100%. In some embodiments, cells processed through the constriction of a microfluidic chip described herein may demonstrate a viability percentage following passage through the constriction of less than or equal to 100%, 95%, 90%, 85%, 80%, or 75%. In some embodiments, cells processed through the constriction of a microfluidic chip described herein may demonstrate a viability percentage following passage through the constriction of more than or equal to 70%, 75%, 80%, 85%, 90%, or 95%.

In some embodiments, cells processed through the constriction of a microfluidic chip described herein, for example in accordance with method 300 may demonstrate a payload delivery percentage of 30-100%. In some embodiments, cells passed through the constriction of a microfluidic chip described herein may demonstrate a payload delivery percentage of less than or equal to 100%, 95%, 90%, 85%, 80%, 75%, 70%, 65%, 60%, 55%, 50%, 45%, 40%, or 35%. In some embodiments, cells passed through the constriction of a microfluidic chip described herein may demonstrate a payload delivery percentage of greater than or equal to 30%, 35%, 40%, 45%, 50%, 55%, 60%, 65%, 70%, 75%, 80%, 85%, 90%, or 95%.

### Flow Characteristics of Microfluidic Chips Provided Herein

Described below are various fluid flow characteristics of fluids that may flow through a microfluidic chip described herein. Specifically, provided below are pressures, shear stresses, shear rates, flow velocities, volumetric flow rates, cell clogging rates, and cell throughputs.

As explained above, microfluidic chips provided herein are designed to squeeze cells by operating under pressure to ensure that the cells of the cell suspension are forced from the upstream region of the fluid flow region to the downstream region of the fluid flow region by way of passing through a constriction such that the membrane of the cells are perturbed. In some embodiments, microfluidic chip 100 may be configured to operate at 1-200 PSI, 10-150 PSI, or 25-100 PSI. In some embodiments, microfluidic chip 100 may be configured to squeeze cells by operating at pressures of less than or equal to 1, 5, 10, 25, 50, 75, 100, 125, 150, or 200 PSI. In some embodiments, microfluidic chip 100 may be configured to squeeze at pressures of greater than or equal to 1, 5, 10, 25, 50, 75, 100, 125, 150, or 200 PSI.

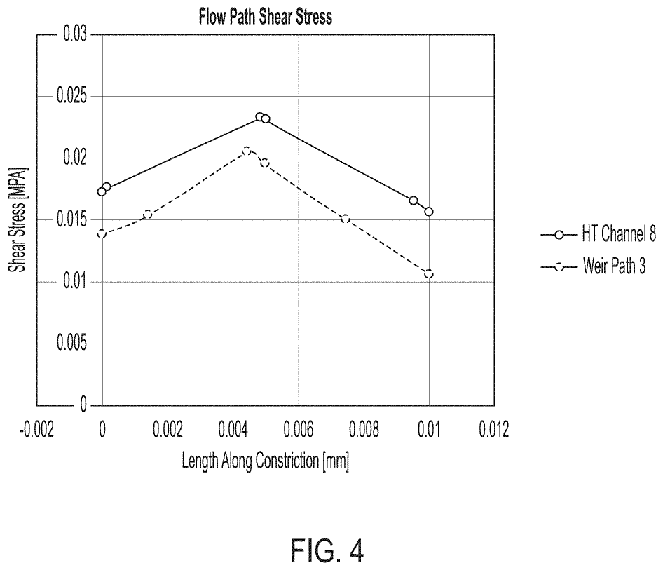

By removing edge effects of the protrusion and/or support pillars of microfluidic chips provided herein, the shear stress of a cell passing through the constriction may be reduced. For example, the maximum shear stress of a cell passing through a constriction of a microfluidic chip provided herein may be 0.001-0.1 MPa, 0.01-0.1 MPa, or 0.01-0.05 MPa. In some embodiments, the maximum shear stress may be less than or equal to 0.1, 0.05, or 0.01 MPa. In some embodiments, the maximum shear stress may be greater than or equal to 0.001, 0.01, or 0.05 MPa. For example, FIG. 4 shows shear stress data for microfluidic chips (e.g., Weir chips) provided herein. As shown, the maximum shear stress of a microfluidic chip provided herein occurs around 0.02 MPa at a distance of approximately 0.004 mm through the constriction.

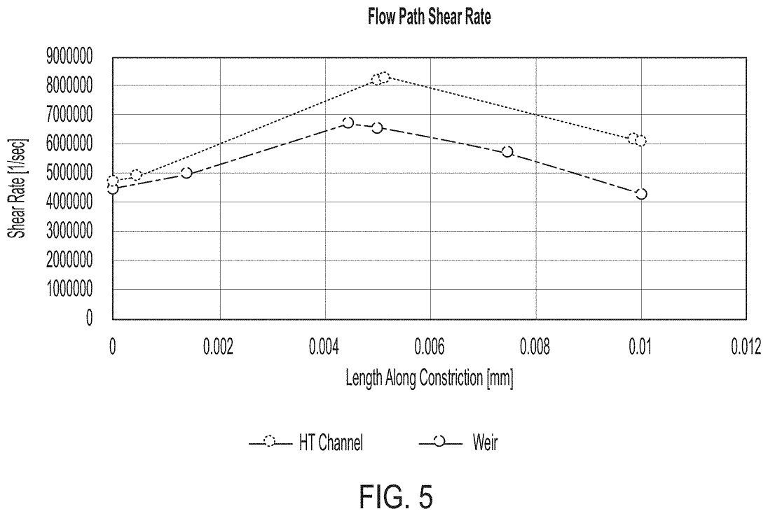

A shear rate of a cell passing through a constriction of a microfluidic chip provided herein may also be decreased by removing edge effects of the protrusion and/or support pillars. For example, the maximum shear rate of a cell passing through a constriction of a microfluidic chip provided herein may be 5-9 (10⁶ Hz) or 6-8 (10⁶ Hz). In some embodiments, the maximum shear rate of a cell passing through a constriction of a microfluidic chip provided herein may be less than or equal to 9, 8.5, 8, 7.5, 7, 6.5, 6, or 5.5 (10⁶ Hz). In some embodiments, the maximum shear rate of a cell passing through a constriction of a microfluidic chip provided herein may be greater than or equal to 5, 5.5, 6, 6.5, 7, 7.5, 8, or 8.5 (10⁶ Hz). For example, FIG. 5 shows shear rate data for microfluidic chips (e.g., Weir chips) provided herein. As shown, the maximum shear rate caused by a microfluidic chip provided herein occurs at approximately 6.75 (10⁶ Hz) and at approximately 0.0045 mm through the length of the constriction.

In some embodiments, microfluidic chip 100 may provide a flow velocity through the constriction of microfluidic chip 100 of 0.5-15 m/sec, 2-12 m/sec, or 3-10 m/sec. In some embodiments, the flow velocity through the constriction is less than or equal to 15, 12, 10, 8, 5, 3, 2, or 1 m/sec. In some embodiments, the flow velocity through the constriction is greater than or equal to 0.5, 1, 2, 3, 5, 8, 10, or 12 m/sec. In some embodiments, microfluidic chip 100 is configured to accept flow of a fluid (e.g., cell suspension) that passes through the constriction without clogging.

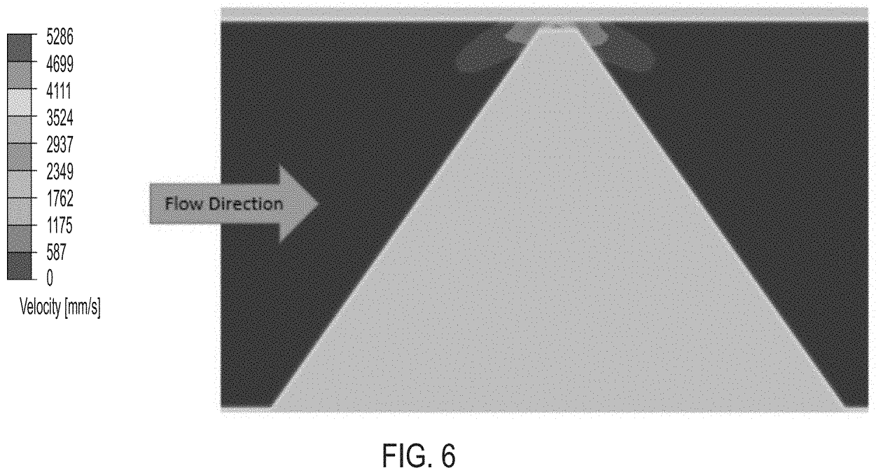

For example, FIG. 6 shows a cross-sectional view of a cell flowing through a constriction formed by a protrusion of a microfluidic chip provided herein. As shown in FIG. 6, the maximum flow rate achieved by a cell through a constriction of a microfluidic chip is 5.286 m/sec.

In some embodiments, microfluidic chip 100 may provide a volumetric flow rate through the constriction of 50-300 mm³/sec, 75-250 mm³/sec, or 100-150 mm³/sec. In some embodiments, the volumetric flow rate through the constriction of microfluidic chip 100 is less than or equal to 300, 250, 200, 150, 100, or 75 mm³/sec. In some embodiments, the volumetric flow rate through the constriction of microfluidic chip 100 is greater than or equal to 50, 75, 100, 150, 200, or 250 mm³/sec.

Microfluidic chips described herein have been designed to minimize cell clogging in the fluid flow regions and/or constriction areas. The number of cells required to clog a microfluidic chip according to embodiments provided herein is dependent upon the cross-sectional area of a constriction, particle/cell diameter, and particle/cell flow rate. This relationship can be defined as:

$${n}_{max} α \frac{A D}{2 {V}_{P}} , A = cross section area , D = particle diameter , \\ V = particle flow rate$$

Based on the cross-sectional area of the particular microfluidic chips described herein, and the particle/cell diameters and velocities used, the number of cells required to clog an area of a microfluidic chip described herein can be defined as:

$${n}_{weir} \approx 6.51 e - 17 \frac{{m}^{2}}{sec}$$

As explained above, microfluidic chips provided herein are specifically designed to have a high-throughput. Due to the particular constriction geometry, the number of cells that can pass through the constriction (and become perturbed by the constriction) is greater than known microfluidic chips for a given unit of time.

### Methods of Etching Microfluidic Chips Described Herein

Various etching methods may be used to etch into the first layer of a microfluidic chip provided herein to form the fluid flow region (including the protrusion(s) and constriction(s)). For example, various etching methods that may be used can include potassium hydroxide (KOH) etching, deep reactive-ion etching (DRIE), and other suitable etching techniques, including various wet etching and dry etching techniques. Microfluidic chips may also be fabricated using molding techniques (e.g., injection molding).

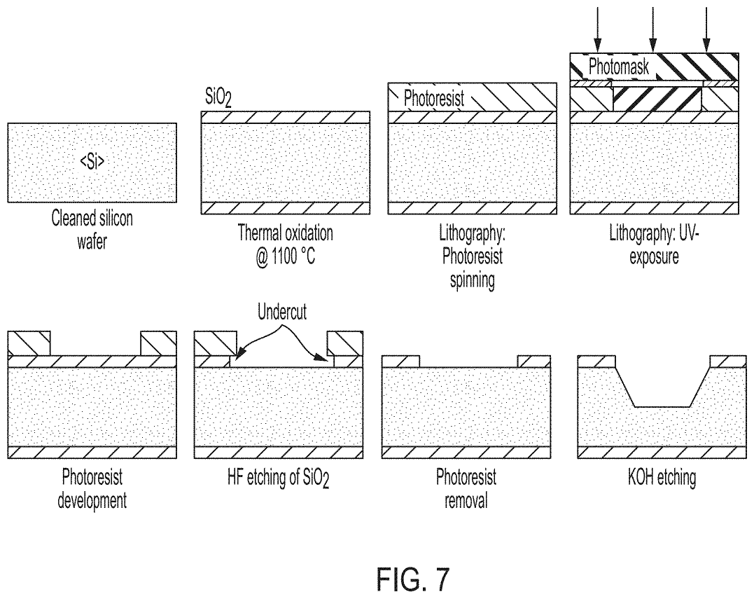

FIG. 7 illustrates an exemplary KOH etching method that may be used to achieve the fluid flow region including the protrusion and constriction of the microfluidic chips described herein. KOH etching is a wet chemical etching process that can be used to create cavities in silicon. The KOH etching process can be controlled by the etching temperature, the percentage of KOH used, the implementation of crystal planes that act as a stop, the degree of anisotropy, the presence of any atomic defects in the silicon, and the presence of other impurities naturally found in the silicon. The etch rate of KOH is a well-understood process, so KOH etching of a silicon layer can achieve consistent etching over a number of microfluidic chips.

As shown in FIG. 7, the silicon wafer is oxidized at 1100 degrees Celsius to form a mask of silicon dioxide on the surface of the silicon wafer. A photoresist is then formed on the surface of the silicon wafer lithography. Specifically, a photoresist spinning method may be used to form the photoresist. After the photoresist is coated on the silicon wafer surface, the coated silicon wafer is exposed to ultraviolet light to remove portions of the photoresist (e.g., the portions under which the fluid flow region will be etched). The photoresist is developed, and an undercut is etched into the silicon dioxide layer using hydrofluoric acid. The photoresist is then removed from the silicon surface, and the silicon is etching using KOH by dipping the masked silicon wafer into a KOH solution. KOH etching achieves the angled protrusion profile, since the KOH etches along the crystal planes of the silicon. This can result in a protrusion profile having an angle of exactly 54.74 degrees (e.g., the angle between an angled side of the protrusion and a base of the protrusion). All other etched surfaces may be flat. If the silicon layer is exposed to the KOH solution for a long period of time, it can result in only rectangular features being formed (and not the 54.74 degree-angled protrusion). In some embodiments, microfluidic chips provided herein may be fabricated using two or more KOH etching processes. For example, a first KOH etch may etch the upstream and/or downstream fluid flow regions, and a second KOH etch may etch the constriction region.

### EXAMPLES

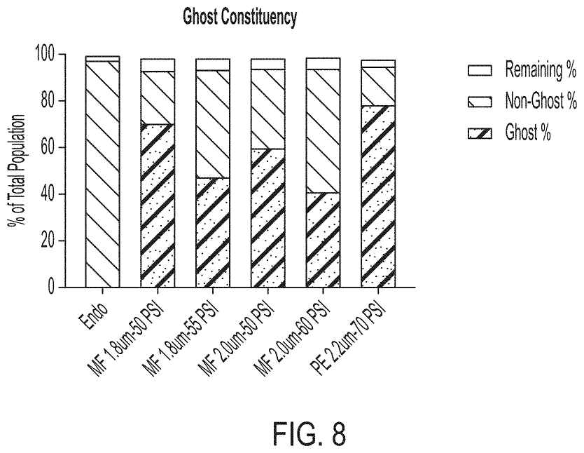

Various microfluidic chip geometries squeezing at different pressures were tested, the results of which are described below in FIGS. 8-16. The microfluidic chips tested are all consistent with the embodiments provided herein. As provided below, each of the microfluidic chips are characterized by a height measurement of the constriction of the chip as measured in a height direction that is perpendicular to a planar surface of a first layer of the chip (z-direction). The microfluidic chips include the following:
a microfluidic chip with a constriction having a height of 1.8 microns, squeezing at 50 PSI;
a microfluidic chip with a constriction having a height of 1.8 microns, squeezing at 55 PSI;
a microfluidic chip with a constriction having a height of 2.0 microns, squeezing at 50 PSI;
a microfluidic chip with a constriction having a height of 2.0 microns, squeezing at 60 PSI; and
a microfluidic chip with a constriction having a height of 2.2 microns, squeezing at 70 PSI.

FIG. 8 shows data from a study evaluating ghost generation in various types of microfluidic chips. The generation of ghosts is advantageous for subsequent biological activity in some embodiments. For example, ghost generation is described in more detail in WO 2017/192785 and WO 2020/15098, both of which are incorporated herein in their entirety. In some embodiments, ghost formation after passing through a constriction is about 5% to about 100%. In some embodiments, ghost formation after passing through the constriction is at least about 5%, 10%, 20%, 30%, 40%, 50%, 60%, 70%, 75%, 80%, 85%, 90%, 95%, or 99%. In some embodiments, ghost formation is measured from about 1.0×10⁻² seconds to at least about 10 days after the cell passes through the constriction.

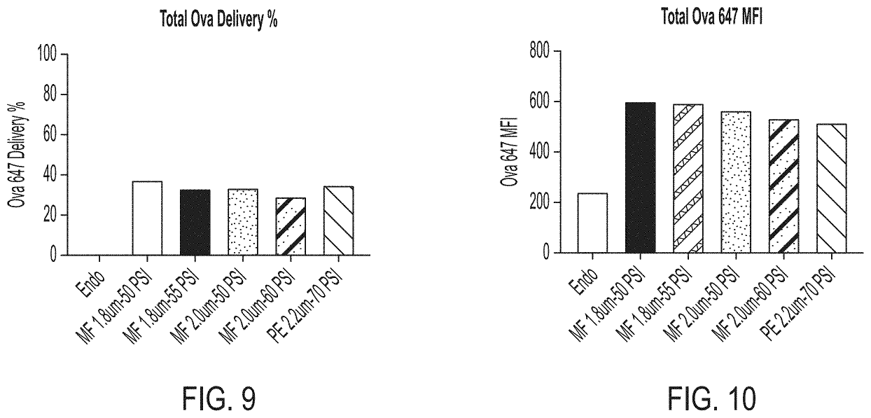

FIGS. 9 and 10 show delivery data for OVA (ovalbumin), which is a specific antigen used as a model protein for studying antigen-specific immune responses. FIG. 9 shows OVA delivery percentages for each of the different microfluidic chips listed above. FIG. 10 shows OVA 647 delivery mean fluorescence intensity (MFI) for each of the different microfluidic chips listed above. OVA 647 is an ovalbumin conjugate that is a protein with a relatively low molecular weight. The fluorescence intensity shows how much payload is delivered into each cell, which can provide a delivery percentage.

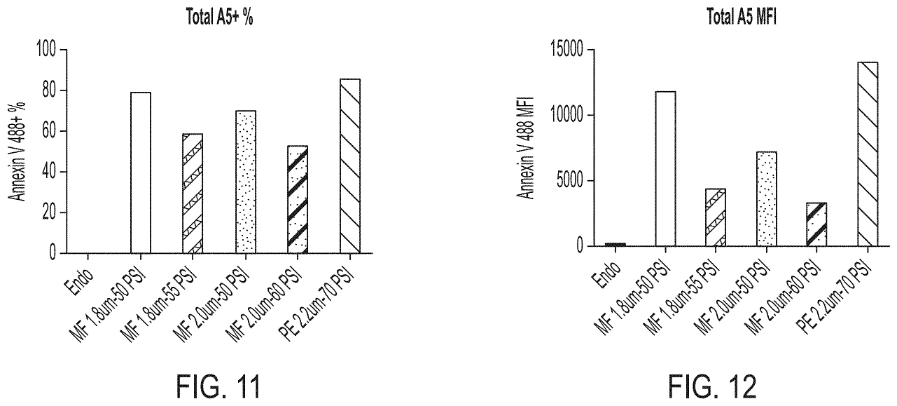

FIGS. 11 and 12 show an analysis of cells stained with annexin V+. FIG. 11 shows data of annexin V+ staining of anucleate cells following passage through each of the different microfluidic chips listed above. The result of annexin V+staining is an indicator that may help quantify the percentage of ghosts in the cell suspension. It may also indicate how intensely the cells were squeezed in the constriction of the specific microfluidic chip tested. FIG. 12 shows data of annexin V+ MFI for each of the different microfluidic chips.

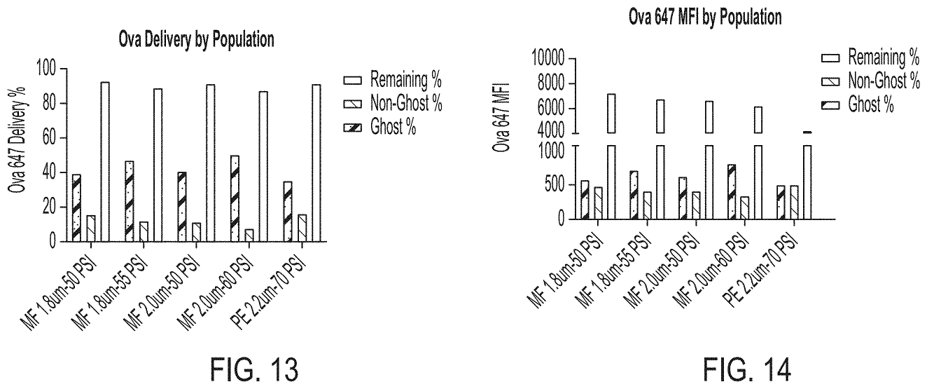

FIGS. 13 and 14 show delivery of OVA to ghosts, non-ghosts and remaining anucleate cells following passage through each of the microfluidic chips provided above. OVA (ovalbumin) is a specific antigen used as a model protein for studying antigen-specific immune responses. FIG. 14 shows MFI for ghosts, non-ghosts and remaining cells following passage of anucleate cells through each of the microfluidic chips provided above.

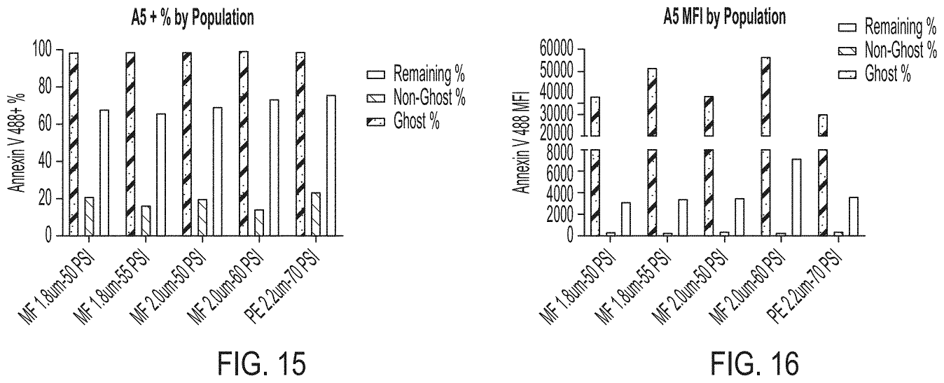

FIGS. 15 and 16 show analysis of cells by staining with annexin V+. FIG. 15 shows total annexin V+ staining percentage for ghosts, non-ghost, and remaining cells following passage of anucleate cells through each of the microfluidic chips provided above. FIG. 16 shows MFI for ghosts, non-ghost, and remaining cells following passage of anucleate cells through each of the microfluidic chips provided above.

The above description sets forth exemplary systems, methods, techniques, parameters, and the like. It should be recognized, however, that such description is not intended as a limitation on the scope of the present disclosure but is instead provided as a description of exemplary embodiments.

### Embodiments

Below is an enumerated listing of certain embodiments. In some embodiments, any one or more of the features of any one or more of the embodiments below may be combined with any one or more of the other embodiments, even if the dependencies of the embodiments do not explicitly indicate that the embodiments may be combined.

Embodiment 1. A microfluidic chip for causing the delivery of a payload to a cell, the chip comprising:
a first layer;
a second layer; and
a fluid flow region between the first layer and the second layer, wherein the chip is configured to accept flow of a cell suspension into the fluid flow region, the cell suspension comprising a plurality of cells,
the first layer comprises a protrusion extending toward the second layer to form a constriction between the protrusion and the second layer, wherein the constriction is configured to cause perturbation of a cell membrane of a cell of the plurality of cells as the cell passes through the constriction.

Embodiment 2. The microfluidic chip of embodiment 1, wherein the first layer comprises silicon.

Embodiment 3. The microfluidic chip of embodiment 1 or 2, wherein the second layer comprises glass.

Embodiment 4. The microfluidic chip of any of embodiments 1-3, wherein the protrusion extends away from an inner surface of the first layer in a height direction perpendicular to the inner surface of the first layer and comprises a proximal end that is adjacent to the inner surface of the first layer.

Embodiment 5. The microfluidic chip of any of embodiments 1-4, wherein the protrusion extends away from an inner surface of the first layer in a height direction perpendicular to the inner surface of the first layer and comprises a distal end of the protrusion that forms the constriction between the distal end of the protrusion and an inner surface of the second layer, wherein a height of the constriction between the distal end of the protrusion and the inner surface of the second layer, as measured in the height direction, is less than or equal to 5 microns.

Embodiment 6. The microfluidic chip of embodiment 5, wherein the height of the constriction is less than a diameter of the cell of the plurality of cells.

Embodiment 7. The microfluidic chip of any of embodiments 1-6, wherein the microfluidic chip comprises an inlet and an outlet, wherein the inlet is positioned at a first end of the chip and the outlet is positioned at a second end of the chip opposite the first end, wherein a distance between the inlet and the outlet extends in a direction perpendicular to an inner surface of the first layer.

Embodiment 8. The microfluidic chip of any of embodiments 4-7, wherein the proximal end of the protrusion has a thickness extending perpendicular to the height direction and extending from an upstream side of the protrusion to a downstream side of the protrusion, wherein the thickness of the proximal end of the protrusion is greater than or equal to 10 microns.

Embodiment 9. The microfluidic chip of any of embodiments 5-8, wherein the distal end of the protrusion has a thickness extending perpendicular to the height direction and extending from an upstream side of the constriction to a downstream side of the constriction, wherein the thickness of the distal end of the protrusion is greater than or equal to 5 microns.

Embodiment 10. The microfluidic chip of any of embodiments 4-9, wherein the protrusion has a length extending perpendicular to the height direction and extending along an interface between an upstream side of the constriction and a downstream side of the constriction, wherein the length is greater than or equal to 0.5 cm.

Embodiment 11. The microfluidic chip of embodiment 10, wherein the interface between the upstream side of the constriction and the downstream side of the constriction that comprises one or more of a curve and an angle.

Embodiment 12. The microfluidic chip of any of embodiments 1-11, wherein the interface between the upstream side of the constriction and the downstream side of the constriction forms a serpentine path.

Embodiment 13. The microfluidic chip of embodiment 12, wherein the serpentine path of the interface is perpendicular to the height direction at all locations along the serpentine path.

Embodiment 14. The microfluidic chip of embodiment 12 or 13, wherein the serpentine path comprises one or more right angles.

Embodiment 15. The microfluidic chip of any of embodiments 1-14, wherein a first dimension of the microfluidic chip extending perpendicular to a height direction that is perpendicular to a planar surface of one or both of the first and second layers is 18-24 mm.

Embodiment 16. The microfluidic chip of any of embodiments 1-15, wherein a second dimension of the microfluidic chip extending perpendicular to a height direction that is perpendicular to a planar surface of one or both of the first and second layers is 8-15 mm.

Embodiment 17. The microfluidic chip of any of embodiments 1-16, wherein a height dimension of the microfluidic chip extending in a height direction that is perpendicular to a planar surface of one or both of the first and second layers is 1000-1500 microns.

Embodiment 18. The microfluidic chip of any of embodiments 1-17, wherein the second layer contacts the first layer at a plurality of support pillars, wherein each support pillar of the plurality of support pillars extends from an inner surface of the first layer to the second layer.

Embodiment 19. The microfluidic chip of any of embodiments 1-18, wherein the first layer has a total height of 500-750 microns as measured in a height direction that is perpendicular to a planar surface of the first layer.

Embodiment 20. The microfluidic chip of any of embodiments 1-19, wherein the first layer has a minimum height of 450-650 microns, wherein the minimum thickness is measured in a height direction that is perpendicular to an inner surface of the first layer and wherein the minimum thickness is measured from an outer surface of the first layer to a nearest inner surface of the first layer that interfaces with the fluid flow region.

Embodiment 21. The microfluidic chip of any of embodiments 1-20, wherein the second layer has a thickness of 450-800 microns as measured in a height direction that is perpendicular to a planar surface of one or both of the first and second layers.

Embodiment 22. The microfluidic chip of any of embodiments 1-21, wherein an uppermost surface of the protrusion comprises a planar surface parallel to an inner surface of the first layer and perpendicular to a height direction in which the protrusion extends from the inner surface of the first layer.

Embodiment 23. The microfluidic chip of any of embodiments 1-22, wherein an uppermost surface of the protrusion comprises a planar surface parallel to an inner surface of the second layer and perpendicular to a height direction in which the protrusion extends from the inner surface of the first layer.

Embodiment 24. The microfluidic chip of any of embodiments 1-23, wherein a side surface of the protrusion extends upwards from an inner surface of the first layer at an angle.

Embodiment 25. The microfluidic chip of embodiment 24, wherein the side surface of the protrusion is angled at 50-60 degrees from the inner surface of the first layer.

Embodiment 26. The microfluidic chip of embodiment 24 or 25, wherein the side surface of the protrusion is angled at 54.7 degrees from the inner surface of the first layer.

Embodiment 27. The microfluidic chip of any of embodiments 1-26, wherein the cell suspension comprises a payload.

Embodiment 28. The microfluidic chip of any of embodiments 1-27, wherein the chip is configured to operate at a pressure of greater than or equal to 10 psi.

Embodiment 29. The microfluidic chip of any of embodiments 1-28, wherein a quotient of a cross-sectional area of the constriction to a perimeter of the constriction is greater than or equal to 0.5 microns.

Embodiment 30. A method of causing the delivery of a payload to a cell, the method comprising:
receiving flow of a cell suspension into a fluid flow region of a microfluidic chip, the cell suspension comprising a plurality of cells;
perturbing a cell membrane of a cell of the plurality of cells by causing the cell to flow through a constriction formed between a protrusion and a second layer of the microfluidic chip, the protrusion extending from an inner surface of a first layer of the microfluidic chip toward the second layer of the microfluidic chip, wherein the perturbation of the cell membrane allows entry of a payload into the cell.

Embodiment 31. The method of embodiment 30, wherein perturbing a cell membrane of a cell of the plurality of cells by causing the cell to flow through a constriction formed between a protrusion and a second layer of the microfluidic chip comprises causing the cell to flow through the constriction at a flow rate of greater than or equal to 0.5 msec.

Embodiment 32. The method of embodiment 30 or 31, wherein the microfluidic chip is configured to operate at a pressure of greater than or equal to 10 psi.

Embodiment 33. The method of any of embodiments 30-32, wherein the cell suspension comprises the payload.

Embodiment 34. The method of any of embodiments 30-33, comprising causing the payload to come into contact with the cell suspension following perturbation of the cell membrane.

Embodiment 35. A method of fabricating a microfluidic chip for causing the delivery of a payload to a cell, the method comprising:
etching into a first layer to form a recessed surface of the first layer and a protrusion of the first layer; and
affixing a second layer to the first layer to form a microfluidic chip having a fluid flow region defined between the recessed surface and the second layer and a constriction defined between the protrusion and the second layer.

Embodiment 36. The method of embodiment 35, wherein etching into a first layer to form a recessed surface of the first layer and a protrusion of the first layer comprises using wet or dry chemical etchants.

Embodiment 37. The method of embodiment 35 or 36, wherein etching into a first layer to form a recessed surface of the first layer and a protrusion of the first layer comprises etching into the first layer to a depth of greater than or equal to 30 microns to define the fluid flow region.

Embodiment 38. The method of any of embodiments 35-37, wherein etching into a first layer to form a recessed surface of the first layer and a protrusion of the first layer comprises etching into the first layer to a depth of less than or equal to 5 microns to form a distal end of the protrusion.

Embodiment 39. The method of any of embodiments 35-38, wherein etching into a first layer to form a recessed surface of the first layer and a protrusion of the first layer comprises etching into the first layer to form a distal end of the protrusion, wherein the distal end of the protrusion has a thickness that extends perpendicular to a height direction that is perpendicular to a planar surface of the first layer, wherein the thickness of the distal end extends from an upstream side of the protrusion to a downstream side of the protrusion, wherein the thickness of the distal end of the protrusion is greater than or equal to 5 microns.

Embodiment 40. The method of embodiment 35, wherein the protrusion has a length extending perpendicular to the height direction and extending along an interface between an upstream side of the constriction and a downstream side of the constriction, wherein the length is greater than or equal to 0.5 cm.

Embodiment 41. The method of embodiment 40, wherein the interface between the upstream side of the constriction and the downstream side of the constriction that comprises one or more of a curve and an angle.

Embodiment 42. The method of embodiment 40 or 41, wherein the interface between the upstream side of the constriction and the downstream side of the constriction forms a serpentine path.

Embodiment 43 The method of embodiment 42, wherein the serpentine path of the interface is perpendicular to the height direction at all locations along the serpentine path.

Embodiment 44. The method of embodiment 42 or 43, wherein the serpentine path comprises one or more right angles.

1. A microfluidic chip for causing the delivery of a payload to a cell, the chip comprising:
a first layer;
a second layer; and
a fluid flow region between the first layer and the second layer, wherein the chip is configured to accept flow of a cell suspension into the fluid flow region, the cell suspension comprising a plurality of cells,
the first layer comprises a protrusion extending toward the second layer to form a constriction between the protrusion and the second layer, wherein the constriction is configured to cause perturbation of a cell membrane of a cell of the plurality of cells as the cell passes through the constriction.

2. The microfluidic chip of claim 1, wherein the first layer comprises silicon.

3. The microfluidic chip of claim 1, wherein the second layer comprises glass.

4. The microfluidic chip of claim 1, wherein the protrusion extends away from an inner surface of the first layer in a height direction perpendicular to the inner surface of the first layer and comprises a proximal end that is adjacent to the inner surface of the first layer.

5. The microfluidic chip of claim 1, wherein the protrusion extends away from an inner surface of the first layer in a height direction perpendicular to the inner surface of the first layer and comprises a distal end of the protrusion that forms the constriction between the distal end of the protrusion and an inner surface of the second layer, wherein a height of the constriction between the distal end of the protrusion and the inner surface of the second layer, as measured in the height direction, is less than or equal to 5 microns.

6. The microfluidic chip of claim 5, wherein the height of the constriction is less than a diameter of the cell of the plurality of cells.

7. The microfluidic chip of claim 1, wherein the microfluidic chip comprises an inlet and an outlet, wherein the inlet is positioned at a first end of the chip and the outlet is positioned at a second end of the chip opposite the first end, wherein a distance between the inlet and the outlet extends in a direction perpendicular to an inner surface of the first layer.

8. The microfluidic chip of claim 4, wherein the proximal end of the protrusion has a thickness extending perpendicular to the height direction and extending from an upstream side of the protrusion to a downstream side of the protrusion, wherein the thickness of the proximal end of the protrusion is greater than or equal to 10 microns.

9. The microfluidic chip of claim 5, wherein the distal end of the protrusion has a thickness extending perpendicular to the height direction and extending from an upstream side of the constriction to a downstream side of the constriction, wherein the thickness of the distal end of the protrusion is greater than or equal to 5 microns.

10. The microfluidic chip of claim 4, wherein the protrusion has a length extending perpendicular to the height direction and extending along an interface between an upstream side of the constriction and a downstream side of the constriction, wherein the length is greater than or equal to 0.5 cm.

11. The microfluidic chip of claim 10, wherein the interface between the upstream side of the constriction and the downstream side of the constriction that comprises one or more of a curve and an angle.

12. The microfluidic chip of claim 1, wherein the interface between the upstream side of the constriction and the downstream side of the constriction forms a serpentine path.

13. The microfluidic chip of claims 12, wherein the serpentine path of the interface is perpendicular to the height direction at all locations along the serpentine path.

14. The microfluidic chip of claim 12, wherein the serpentine path comprises one or more right angles.

15. The microfluidic chip of claim 1, wherein a first dimension of the microfluidic chip extending perpendicular to a height direction that is perpendicular to a planar surface of one or both of the first and second layers is 18-24 mm.

16. The microfluidic chip of claim 1, wherein a second dimension of the microfluidic chip extending perpendicular to a height direction that is perpendicular to a planar surface of one or both of the first and second layers is 8-15 mm.

17. The microfluidic chip of claim 1, wherein a height dimension of the microfluidic chip extending in a height direction that is perpendicular to a planar surface of one or both of the first and second layers is 1000-1500 microns.

18. The microfluidic chip of claim 1, wherein the second layer contacts the first layer at a plurality of support pillars, wherein each support pillar of the plurality of support pillars extends from an inner surface of the first layer to the second layer.

19. The microfluidic chip of claim 1, wherein the first layer has a total height of 500-750 microns as measured in a height direction that is perpendicular to a planar surface of the first layer.

20. The microfluidic chip of claim 1, wherein the first layer has a minimum height of 450-650 microns, wherein the minimum thickness is measured in a height direction that is perpendicular to an inner surface of the first layer and wherein the minimum thickness is measured from an outer surface of the first layer to a nearest inner surface of the first layer that interfaces with the fluid flow region.

21. The microfluidic chip of claim 1, wherein the second layer has a thickness of 450-800 microns as measured in a height direction that is perpendicular to a planar surface of one or both of the first and second layers.

22. The microfluidic chip of claim 1, wherein an uppermost surface of the protrusion comprises a planar surface parallel to an inner surface of the first layer and perpendicular to a height direction in which the protrusion extends from the inner surface of the first layer.

23. The microfluidic chip of claim 1, wherein an uppermost surface of the protrusion comprises a planar surface parallel to an inner surface of the second layer and perpendicular to a height direction in which the protrusion extends from the inner surface of the first layer.

24. The microfluidic chip of claim 1, wherein a side surface of the protrusion extends upwards from an inner surface of the first layer at an angle.

25. The microfluidic chip of claim 24, wherein the side surface of the protrusion is angled at 50-60 degrees from the inner surface of the first layer.

26. The microfluidic chip of claim 24, wherein the side surface of the protrusion is angled at 54.7 degrees from the inner surface of the first layer.

27. The microfluidic chip of claim 1, wherein the cell suspension comprises a payload.

28. The microfluidic chip of claim 1, wherein the chip is configured to operate at a pressure of greater than or equal to 10 psi.

29. The microfluidic chip of claim 1, wherein a quotient of a cross-sectional area of the constriction to a perimeter of the constriction is greater than or equal to 0.5 microns.

30. A method of causing the delivery of a payload to a cell, the method comprising:
receiving flow of a cell suspension into a fluid flow region of a microfluidic chip, the cell suspension comprising a plurality of cells;
perturbing a cell membrane of a cell of the plurality of cells by causing the cell to flow through a constriction formed between a protrusion and a second layer of the microfluidic chip, the protrusion extending from an inner surface of a first layer of the microfluidic chip toward the second layer of the microfluidic chip, wherein the perturbation of the cell membrane allows entry of a payload into the cell.

31. The method of claim 30, wherein perturbing a cell membrane of a cell of the plurality of cells by causing the cell to flow through a constriction formed between a protrusion and a second layer of the microfluidic chip comprises causing the cell to flow through the constriction at a flow rate of greater than or equal to 0.5 m/sec.

32. The method of claim 30, wherein the microfluidic chip is configured to operate at a pressure of greater than or equal to 10 psi.

33. The method of claim 30, wherein the cell suspension comprises the payload.

34. The method of claim 30, comprising causing the payload to come into contact with the cell suspension following perturbation of the cell membrane.

35. A method of fabricating a microfluidic chip for causing the delivery of a payload to a cell, the method comprising:
etching into a first layer to form a recessed surface of the first layer and a protrusion of the first layer; and
affixing a second layer to the first layer to form a microfluidic chip having a fluid flow region defined between the recessed surface and the second layer and a constriction defined between the protrusion and the second layer.

36. The method of claim 35, wherein etching into a first layer to form a recessed surface of the first layer and a protrusion of the first layer comprises using wet or dry chemical etchants.

37. The method of claim 35, wherein etching into a first layer to form a recessed surface of the first layer and a protrusion of the first layer comprises etching into the first layer to a depth of greater than or equal to 30 microns to define the fluid flow region.

38. The method of claim 35, wherein etching into a first layer to form a recessed surface of the first layer and a protrusion of the first layer comprises etching into the first layer to a depth of less than or equal to 5 microns to form a distal end of the protrusion.

39. The method of claim 35, wherein etching into a first layer to form a recessed surface of the first layer and a protrusion of the first layer comprises etching into the first layer to form a distal end of the protrusion, wherein the distal end of the protrusion has a thickness that extends perpendicular to a height direction that is perpendicular to a planar surface of the first layer, wherein the thickness of the distal end extends from an upstream side of the protrusion to a downstream side of the protrusion, wherein the thickness of the distal end of the protrusion is greater than or equal to 5 microns.

40. The method of claim 35, wherein the protrusion has a length extending perpendicular to the height direction and extending along an interface between an upstream side of the constriction and a downstream side of the constriction, wherein the length is greater than or equal to 0.5 cm.

41. The method of claim 40, wherein the interface between the upstream side of the constriction and the downstream side of the constriction that comprises one or more of a curve and an angle.

42. The method of claim 40, wherein the interface between the upstream side of the constriction and the downstream side of the constriction forms a serpentine path.

43. The method of claim 42, wherein the serpentine path of the interface is perpendicular to the height direction at all locations along the serpentine path.

44. The method of claim 42, wherein the serpentine path comprises one or more right angles.
